(19) United States
(12) Patent Application Publication
(10) Pub. No.: US 2018/0197626 A1
(43) Pub. Date: Jul. 12, 2018

(54) Medical Personal Data Card And System

(71) Applicant: F. Maury Matthews, Brentwood, MO (US)

(72) Inventor: F. Maury Matthews, Brentwood, MO (US)

(21) Appl. No.: 15/867,076

(22) Filed: Jan. 10, 2018

Related U.S. Application Data
(60) Provisional application No. 62/444,447, filed on Jan. 10, 2017.

(51) Int. Cl. G16H 10/65; G06K 19/06; G06K 19/07

(52) U.S. Cl. CPC G16H 10/65; G06K 19/0723; G06K 19/06187

(57) ABSTRACT

A medical personal data card system (10) comprises a card (C) containing a person's medical information and a device (R) for accessing the information stored on the card. The information is encoded information, stored in an encrypted format, in compliance with HIPAA regulations. The card and the information stored on it are accessed and used by medical personnel under appropriate circumstances.

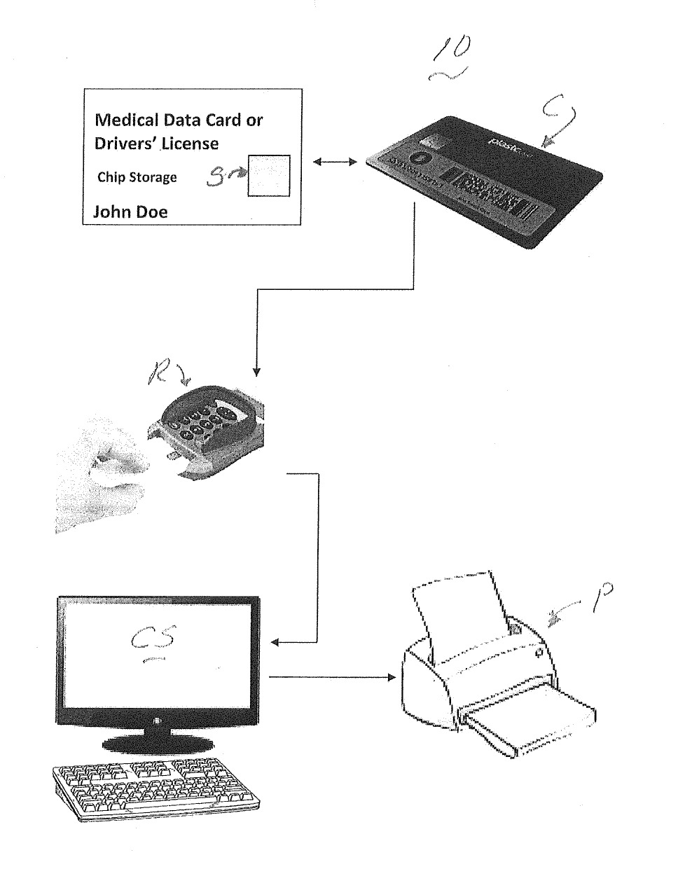

### REFERENCE TO RELATED APPLICATIONS

This application is based on and claims the benefit of U.S. provisional patent application 62/444,447 filed Jan. 10, 2017.

### BACKGROUND OF THE INVENTION

The need for, and the process of, digitizing individual medical records has been recognized for over 20 years and has been established as either EMR (Electronic Medical Records) or EHR (Electronic Health Records). EMRs are digitized versions of paperwork in a clinician's office and contain the medical and treatment history of patients at a practice. EHRs provide more information than EMRs as they focus on a patient's total health; not just the standard clinical data, but a broader view of the health care being provided. EHRs facilitate sharing data outside the particular practice with other health care providers such as laboratories and specialists. Therefore, EHRs record information from all the clinicians involved in the patient's care.

As a part of the American Recovery and Reinvestment Act, all public and private healthcare providers and other eligible professionals (EP) were required to adopt and demonstrate “meaningful use” of electronic medical records (EMR) by Jan. 1, 2014 in order to maintain their existing Medicaid and Medicare reimbursement levels. Since that date, the use of electronic medical and health records has spread worldwide and shown its many benefits to health organizations everywhere. The industry that evolved to design, transfer, and sell medical facilities the wherewithal to accomplish this mandated transition has, unfortunately, not fully developed a system that is transparent and easily shared with medical facilities utilizing differing digital formats.

The result is that there is an impediment to effective and timely transmission of both EMR and EHR data between institutions in a patient emergency. This critical need has driven the necessity for a device; i.e., a Personal Medical Data Card (“MDC”) that is able to immediately provide detailed medical information in an emergency condition (EMT) or in a hospital where such medical history and diagnostic data can shortcut unnecessary medical testing (CATSCANS, MRIs or other invasive procedures). Since the MDC also provides the information of the patient's primary physician, actions otherwise taken in a vacuum now have a resource(s) to provide guidance for treatment.

The initiator of the Medical Data card is the patient's primary physician as they act as the patient's referring conduit to other medical specialists. Should the patient not have a primary physician, the clinic, hospital, or other Medical authorities shall become the initiator of the MDC.

Accordingly, this invention relates to a person's medical information; and, more particularly, to a medical personal data card system containing the person's medical information, which can be carried by the person, which can accessed and used by authorized medical personnel under appropriate circumstances, and which complies with the Health Insurance Portability and Accountability Act or HIPAA.

As is well-known in the medical field, HIPAA includes privacy provisions that prevent protected patient information from being communicated from one party, person, or entity, to another unless certain guidelines are followed.

The “Authorized individual accessing the Medical History of the patient must enter an authorizing code (algorithm) containing the day's date and time, vehicle or facility code and the users personal ID number along with a biometric such as a fingerprint. This discrete code and biometric, Authorizing ID (“Personal ID”) is a dongle maintained by the hospital or other authorizing agent whether an EMT, Fire Department, Hospital or other certified agency dealing with emergences and or physical/medial crisis as determined by the State, County or Municipality. The Personal ID determines the authority to access the patient's Medical Data Card and the level allowed.

The “chip” is partitioned so that the authorized user can only access information pertinent to the activity undertaken. As an example, the first responders can access, name, age, medical conditions (Angina, Diabetes, Hyper-Tension, Emphysema, Coronary Heart Disease and other conditions that foreknowledge assists in appropriate medical measures). Other partitions are only accesses by the appropriate personnel in the emergency room and/or hospital).

The chip also lists all active and recent prescriptions drugs (and allergies or adverse reactions) and their dosage and well as ongoing medical treatment for diseases such as cancer (Chemotherapy and/or radiation) and/or other medical treatments.

While patient confidentiality is important, the regulations imposed under HIPAA can, in some circumstances, act as a hindrance when there is a need for medical personnel to access vital patient information. A particular example of such a situation would be an emergency responder at an accident scene needing information to treat a victim so to insure, for example, that the person would not have a negative reaction to an injection.

Obviously, these circumstances include medical emergencies, particularly when the person is under emergency care by an EMT or other qualified and trained person or is in a location distant from their home. However, in more mundane settings, the need to readily access vital information may also arise. For example, a person is visiting their doctor but, for some reason, the doctor's internal system breaks down and prevents the doctor or his or her staff from accessing the person's medical information. While not necessarily an emergency, it is certainly an inconvenience to both the person and the doctor.

The devices rely on technology used often in “smart cards” by the credit card industry to emboss the card with the patient's name and read/write pertinent information regarding the patient's medical conditions, history, including tests, x-rays, MRIs, and or CAT Scans and other images along with prescription drugs, organ donation, contact individuals and written documents such as medical directives and living wills.

### BRIEF SUMMARY OF THE INVENTION

The present disclosure is directed to a plastic (PVC type) card with a read/write chip which can be carried by someone on their person (e.g., on a bracelet or necklace, lanyard or on their body) and contains the person's medical information in compliance with HIPAA regulations.

The disclosure is also directed to a device that is coded or encrypted allowing access to the person's medical information through a reader device that requires the individual accessing the coded card using the reader device to be an authorized individual, again in compliance with HIPAA regulations, particularly during emergency situations when the card holder is either away from his or her home because of travel, or is in transit to an emergency room at a local hospital and is not conscious, lucid, or otherwise conversant. Timely accessibility to the information allows potential lifesaving treatment to be performed both during transit to an emergency room or in an emergency room by emergency medical technicians (EMTs) or other first responders at the hospital.

The chip also provides contact information for the patient's family and medical professional who normally treat him or her, as well as information included in a Living Will, Organ Donor release or the like.

The card and devices are respectively affordable by the individual and by medical institutions.

### BRIEF DESCRIPTION OF THE DRAWING

The sole drawing FIGURE illustrates a medical personal data card and a system for its use.

### DETAILED DESCRIPTION OF INVENTION

The following detailed description illustrates the invention by way of example and not by way of limitation. This description clearly enables one skilled in the art to make and use the invention, and describes several embodiments, adaptations, variations, alternatives and uses of the invention, including what is presently believed to be the best mode of carrying out the invention. Additionally, it is to be understood that the invention is not limited in its application to the details of construction and the arrangement of components set forth in the following description or illustrated in the drawings. The invention is capable of other embodiments and of being practiced or carried out in various ways. Also, it will be understood that the phraseology and terminology used herein is for the purpose of description and should not be regarded as limiting.

As shown in the drawing, the MDC card is created using a device connected to a medical IT system that holds the patient's Medical Records containing pertinent medical tests, x-rays, MRIs etc. These records are written to the MDC's chip in “plain English”. The Card can be embossed with the Patient's name, DOB, and address; or, the DOB and address can be written to a magnetic strip.

As shown in the drawing FIGURE, a medical personal data card system 10 includes a card C which is made of a plastic or other durable material and has a size and shape corresponding to that of a conventional credit card. The card may have a magnetic strip (not shown) on its back, and storage chip S incorporated on it. A person's medical information is encoded, in an encrypted format, on the strip or chip. The information stored on the card can include both data and text.

The information is stored on the card in accordance with HIPAA regulations, the strip or chip having a storage capacity of at least one gigabyte. Those skilled in the art will understand that while card C can be a standalone card; the strip or chip can be incorporated on, for example, the person's driver license or other government issued form of identification.

In use, card C can be read in a number of convenient ways. These include a chip reader R, an optical scanner (not shown), or a magnetic strip reader (also not shown). When read by an authorized individual, the data contained on the strip or chip is provided to, for example, a computer where the patient information is presented on a screen CS for viewing, or provided to a printer P by which a hard copy of the information is made. Typically the most recent medical information about the person is accessed first and is in a first partition accessible by an authorized individual. Information in other(s) partitions cannot be accessed by an individual of a “lower” authority.

Conveniently, the card or chip can be provided by a medical alert (MEDALERT) provider and is worn by the person. The information on the chip is then used by first responders and EMTs for treating the person during a medical emergency.

It will be understood that, pursuant to the procedure or test performed on the person, or to which the person is subjected, the information on the card can be updated. This may occur after each procedure or test, or the completion of all procedures and tests.

In view of the above, it will be seen that the several objects and advantages of the present disclosure have been achieved and other advantageous results have been obtained.

Having thus described the invention, what is claimed and desired to be secured by Letters Patent is:

1. A medical personal data card system comprising a card containing a person's medical information and a device for accessing the information stored on the card, the information being encoded information stored in an encrypted format in compliance with HIPAA regulations, the card and the information stored on it being accessed and used by medical personnel under appropriate circumstances.

2. The system of claim 1 in which the card can be carried by the patient on his or her person.

3. The system of claim 1 in which the card can be the person's driver license or other government issued form of identification.

4. The system of claim 1 in which the information stored on the card is stored on one of a magnetic strip or chip.

5. The system of claim 4 further including a card reader for accessing the information stored on the card in a HIPAA compliant manner.

6. The system of claim 5 in which the information can be viewed on a data screen and printed on a printer for producing a hard copy of the person's medical information.

7. The system of claim 1 in which the information on the card can be updated after tests and procedures to which the patient is subjected are completed.
